(19) United States
(12) Patent Application Publication
(10) Pub. No.: US 2011/0027375 A1
(43) Pub. Date: Feb. 3, 2011

(54) USE OF LANTHANIDE-BASED NANOPARTICLES AS RADIOSENSITIZING AGENTS

(76) Inventors: Olivier Tillement, Fontaines Saint Martin (FR); Stéphane Roux, Villeurbanne (FR); Pascal Perriat, Lyon (FR); Géraldine Leduc, Crolles (FR); Céline Mandon, Lyon (FR); Brice Mutelet, Villeurbanne (FR); Christophe Alric, Bron (FR); Claire Billotey, Lyon (FR); Marc Janier, Lyon (FR); Cédric Louis, Les Cotes D'Arey (FR)

Correspondence Address: CLARK & BRODY, 1700 Diagonal Road, Suite 510, Alexandria, VA, 22314, US

(21) Appl. No.: 12/738,191

(22) PCT Filed: Oct. 14, 2008
(86) PCT No.: PCT/FR08/51860

§ 371 Date: Oct. 18, 2010

(30) Foreign Application Priority Data
Oct. 16, 2007 (FR) 0758348

(51) Int. Cl. A61K 9/14; A61K 33/24; A61P 35/00

(52) U.S. Cl. 424/497; 424/617; 977/773

(57) ABSTRACT

The invention relates to the use of nanoparticles with dimensions comprised between 1 and 50 nm, at least one portion of which consists of at least one oxide and/or one oxohydroxide of at least one lanthanide, said nanoparticles:
either consisting of at least one oxide and/or one oxohydroxide of at least one lanthanide,
or in the form of nanoparticles comprising a core consisting of at least one oxide and/or one oxohydroxide of at least one lanthanide, and a coating consisting of polysiloxane, with possibly organic molecules grafted at the surface or comprised inside it,
as a radio-sensitizing agent in the making of an injectable composition intended to improve the efficiency of the treatment of a tumor by X or gamma irradiations.

It also relates to nanoparticles particularly suitable for the use above, consisting of a core consisting of at least one oxide and/or one oxohydroxide of at least one lanthanide and of a coating in polysiloxane comprising 1 to 5 silicon atoms per lanthanide and at least 10% of the silicon atoms of which are bound to hydrophilic organic molecules with molar masses of less than 450 g/mol.

The invention relates to novel agents intended to increase the efficiency of radiotherapies.

More specifically, the invention aims at the use of nanoparticles with a high concentration of lanthanide oxides, as radio-sensitizers.

X or gamma radiations are widely used for treating tumors. Nevertheless, such radiations are not generally specific of tumors, and significant doses of radiations may also be given to healthy tissues during the treatments.

The use of radiosensitizers has been proposed for many years. A radiosensitizing compound is a compound which acts in combination with the radiation for inducing a more efficient response and for increasing therapeutic efficiency. Compounds which include in their internal structure heavy elements with high atomic number and which directly interact with the radiation by increasing its interaction probability, generating damages to the targeted cells are generally targeted.

A greater portion of the irradiated energy is then absorbed and locally deposited around radiosensitizers and may produce secondary electrons, Auger electrons, Compton electrons, ionizations, photons, free radicals for example, or even simply a increase in heat.

For cancer therapies, one goal is to locally increase the dose at the tumor; the radio-sensitizers thus have to be preferentially accumulated in tumors relatively to healthy tissues.

The increase in the absorbed X irradiation dose because of the use of elements with high atomic number, has been known for more than 50 years and is considered as potentially of interest for increasing therapeutic effects in vitro (H. Matsudaira et al. <<Iodine contrast medium sensitizes cultured mammalian cells to X-rays>>, Rad. Res. 84: 144-148, 1980). Specific calculations and measurements of the effect have been performed by Das et al. (Das et al. <<Backscatter dose perturbation in kilovoltage photon beams at high atomic number interfaces>> Med. Phys. 22:767-773, 1995).

Molecular compounds using heavy elements have been proposed.

For example carboplatin, cisplatin and oxiplatinum have demonstrated good efficiencies (C. Diointe et al., <<Comparisons of carboplatin and cisplatin as potentiators of 5-fluorouracil and radiotherapy in mouse L1210 leukemia model>> Anticancer Res. 22, 721-725, 2002).

Compounds based on lanthanides such as gadolinium or lutetium, have also been proposed. Two metallotexaphyrins, motexafine gadolinium (Gd-Tex) and motexafine lutetium (Lu-Tex) are the subject of significant developments (E. Kowinsky, Oncology 13 (1999) 61).

Gd-Tex in particular has the capacity of inducing both a rise in the MRI contrast and the effect of the delivered dose.

However, it proves to be difficult to concentrate these agents in a tumor because of their molecular nature which favors rapid elimination and extravascular diffusion. This characteristic opposes efficient application of these therapeutic agents because with their large mobility it is not possible to attain a sufficient difference between the amount of agents in the tumor and that in the healthy tissues. This quasi-homogeneous distribution is not without any dangers since it affects both the healthy tissues and the tissues to be treated (without including the toxicity inherent to some of these therapeutic agents).

The use of finely divided solids has been proposed as radiosensitizing agents for compensating the insufficiencies reported with molecular agents. The use of metal, in particular gold, seemed to be rapidly interesting. Thus, Herold et al. suggested in 2000 the use of gold microbeads (D. Herold et al., <<Gold microspheres a selective technique for producing biologically effective dose enhancement>> Int. J. Rad. Biol. 76, 1357-1364, 2000): the mobility and the dispersion of the powders have however rapidly posed a problem.

More recently, Hainfeld et al. suggested the use of metal nanoparticles for enhancing the therapeutic effect (WO 2400/112590).

In the international application above, the use of metal nanoparticles consisting of heavy elements with large atomic number Z, in particular gold, is suggested. By using small particles, it is possible to expect good dispersion of the radiosensitizers. On the other hand, with the high density coupled to the large atomic number of the element, good absorption of the radiations may be expected.

This work was followed by theoretical studies confirming the radiosensitizing effect of gold within the scope of X-ray treatment and in different current situations of treatment (S. H. Cho, Estimation of tumor dose enhancement due to gold nanoparticles during typical radiation treatments: a preliminary Monte Carlo Study, Phys. Med. Biol. 50 (2005) N163).

If the mechanisms brought into play in the therapeutic effect are not yet exactly understood, the use of metal nanoparticles seems very interesting. However, it remains limited to noble metals, often valuable and very expensive, since the use of other elements in metal fowl risks causing undesirable oxidation effects with are difficult to control.

French patent application FR 2 877 571 describes nanoparticles provided with an intracellular targeting element, their preparations and their uses. The nanoparticles described in this document are composite nanoparticles provided with an intracellular targeting element capable of generating a response to electromagnetic or neutron excitations. These nanoparticles comprise a core comprising at least one inorganic compound and possibly one or several other organic compounds and may be activated in vivo for marking or altering cells, tissues or organs. This document cites many inorganic oxides and hydroxides as constituents of the core, but by no means contemplates the specific used of lanthanide oxides or the possibility, provided this element is selected as essential constituent of the nanoparticle, of not resorting to a targeting molecule exposed at the surface of the nanoparticle.

Nanoparticles base on lanthanide oxides and in particular based on gadolinium oxides have already been described in many documents from the literature. (For example: M. Engström, A. Klasson, H. Pedersen, C. Vahlberg, P. O. Käll, K. Uvdal, High proton relaxivity for gadolinium oxide nanoparticles, Magn. Reson. Mater. Phys. (2006) 19, 180-186 and Hybrid gadolinium oxide nanoparticles: Multimodal contrast agents for in vivo imaging, H. Am. Chem. Soc. 2007, 129, 5076-5084 show how the latter may be used as MRI contrast agents).

In the International Application WO 2005/088314 hybrid nanoparticles have in particular been described, comprising a core consisting of a rare earth oxide, possibly doped with a rare earth or an actinide or a mixture of rare earths or else a mixture of a rare earth and actinides in which at least 50% of the metal ions are rare earth ions, a coating around this core, consisting in majority of functionalized polysiloxanes and at least one biological ligand grafted by a covalent bond to the polysiloxane coating. The nanoparticles described in this document are essentially used for applications as probes for detecting, tracking and quantifying biological systems.

It was now discovered that nanoparticles, the structure of which may in certain cases match that of the particles described in the International Application WO 2005/088314, find particularly interesting applications as radio-sensitizing agents in a radiotherapy treatment by means of X or gamma rays. The present invention therefore relates to novel particularly efficient radiosensitizing agents which do not require mandatory resorting to biomolecules at the surface for targeting the cells.

Moreover, it appeared to the inventors of the present invention that the use of such nanoparticles led to products, both more economical than those of the prior art based on gold and also much easier to handle than these metal products which are often very reactive and oxidizable within the biological medium (if these are not noble metals) and more difficult to functionalize in a stable way via covalent bonds. Further, these metal products are difficult to synthesize into alloys of heavy elements and stable combinations seem to be very delicate to make.

More specifically, the invention proposes novel radiosensitizing agents as nanoparticles, the size of which is comprised between 1 and 50 nm, these particle may either consist exclusively of one or more oxides or oxohydroxides of lanthanides or consist in a particularly advantageous way of a core consisting of lanthanide oxides or oxohydroxides and of a either inorganic or mixed organic-inorganic coating, consisting of polysiloxane, with possibly organic molecules grafted at the surface or comprised inside it.

By using such nanoparticles, it is possible, by acting on the composition of these nanoparticles, to simultaneously obtain a significant number of additional advantages which will emerge from the whole of the description and from the examples and this, with particles for which the making proves to be particularly simple and economical, as compared with that of particles based on valuable metals described in the prior art or with that requiring the presence of targeting materials.

Thus, according to an essential feature of its first object, the invention relates to the use of nanoparticles with dimensions comprised between 1 and 50 nm, at least one portion of which consists of at least one oxide and/or oxohydroxide of at least one lanthanide, said nanoparticles:
either consisting of at least one oxide and/or one oxohydroxide of at least one lanthanide,
or as nanoparticles comprising a core consisting of at least one oxide and/or one oxohydroxide of at least one lanthanide, and a coating consisting of polysiloxane, with possible organic molecules grafted at the surface or comprised in it,

as a radio-sensitizing agent in the making of an injectable composition intended to improve the efficiency of the treatment of a tumor by X or gamma irradiations.

According to a second object, the invention relates to nanoparticles, as novel products, proving to be particularly interesting for applying the invention because they are in the form of a core and of a very particular coating.

These nanoparticles are characterized in that they consist:
of a core consisting of at least one oxide and/or one oxohydroxide of at least one lanthanide defined in the same way as in the first object, said core having a size comprised between 1 and 2 nm, and
of a coating of said core in polysiloxane comprising 1-5 silicon atoms per lanthanide and at least 10% of the silicon atoms of said polysiloxane are covalently bound to hydrophilic organic molecules with molar masses of less than 450 g/mol, preferably selected from organic molecules including alcohol or carboxylic acid or amine or amide or ester or ether-oxide or sulfonate or phosphonate or phosphinate functions.

The invention also relates to injectable compositions containing the novel nanoparticles of the invention.

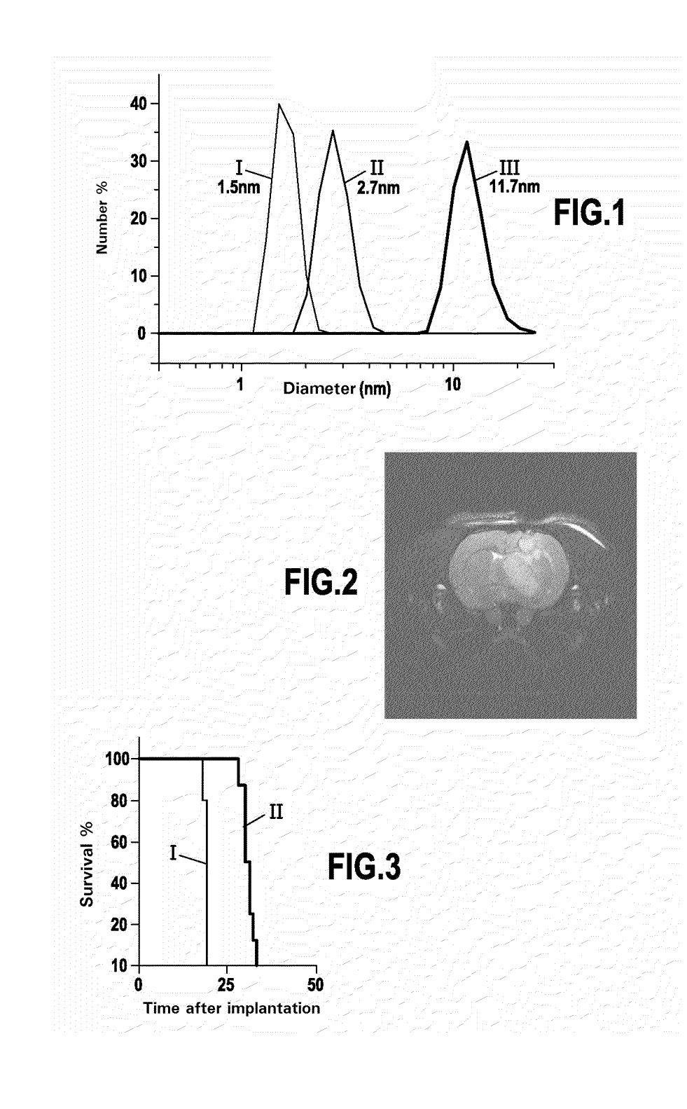

Other features and advantages of the invention will become apparent in the following description as well in the examples referring to FIGS. 1-8:

FIG. 1 given as a reference to Example 1 illustrates the measurement by photon correlation spectroscopy of the size and size distribution of the nanoparticles,

FIG. 2 illustrates the weighted T2 images of the brain of a rat bearing an implanted tumor according to Example 6,

FIG. 3 illustrates the survival percentage of rats bearing a brain tumor implanted according to Example 6, after a treatment according to Example 10,

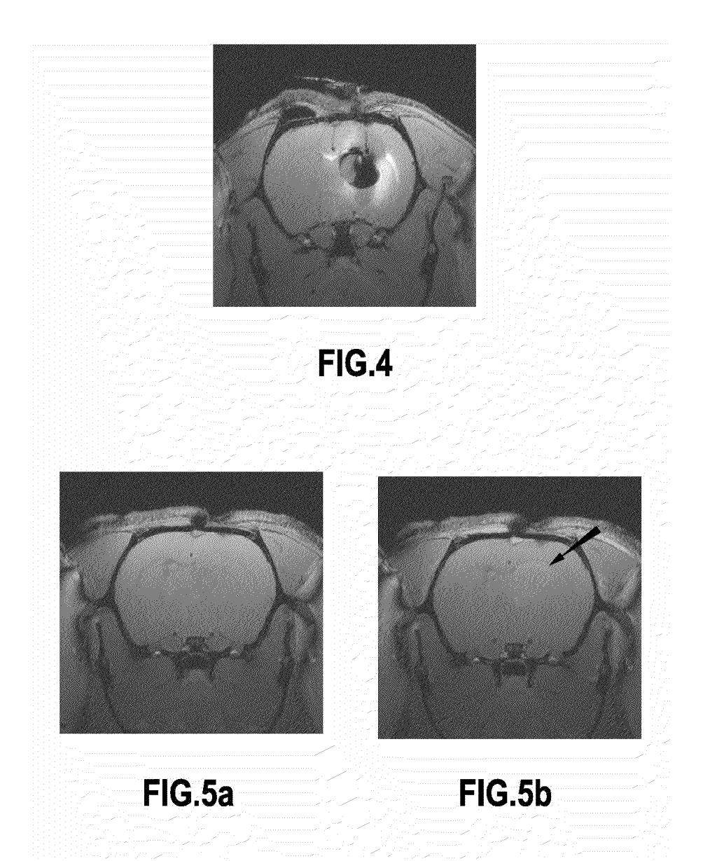

FIG. 4 illustrates the weighted images of the brain of a rat bearing a tumor implanted according to Example 6, after a treatment according to Example 7,

FIG. 5 illustrates the weighted images of the brain of a rat before (FIG. 5a) and after (FIG. 5b) a treatment according to Example 9,

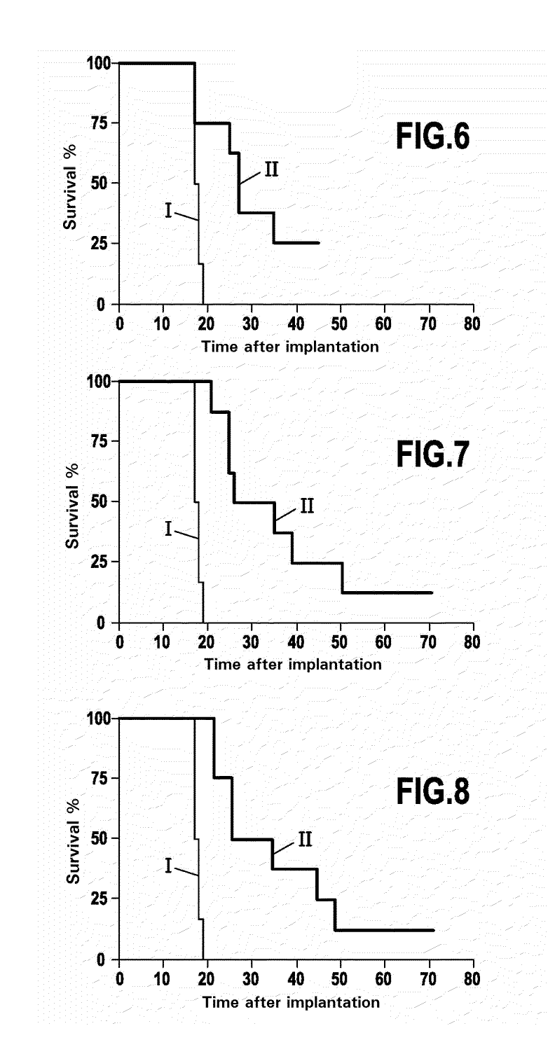

FIG. 6 illustrates the survival percentage of rats bearing a brain tumor implanted according to Example 6, after a treatment according to the comparative Example 12,

FIG. 7 illustrates the survival percentage of rats bearing a brain tumor implanted according to Example 6, after a treatment according to Example 14,

FIG. 8 illustrates the survival percentage of rats bearing a brain tumor implanted according to Example 6, after a treatment according to Example 15.

The nanoparticles used according to the invention have dimensions comprised between 1 and 50 nm and may exclusively consist of at least one oxide and/or one oxohydroxide of at least one lanthanide or may appear in a coated form.

They may exclusively consist of a core comprising at least one oxide or oxohydroxide of at least one lanthanide or a mixture of oxides, a mixture of oxohydroxides or a mixture of oxide and oxohydroxide of at least one lanthanide or may appear as a core surrounded by a coating. In this case, the core comprises at least one oxide or oxohydroxide of at least one lanthanide or a mixture of oxides, oxohydroxides or oxide and oxohydroxide of at least one lanthanide. According to an alternative embodiment, the particles exclusively consist of a lanthanide oxide, a mixed lanthanide oxide (i.e. of at least two lanthanides), of a lanthanide oxohydroxide, of a mixed lanthanide oxohydroxide (i.e. of at least two lanthanides) or one of their mixtures. Also, in the case when the particles according to the invention are found in coated form, according to an alternative embodiment, the core of the particles exclusively consists of a lanthanide oxide, a mixed lanthanide oxide (i.e. of at least two lanthanides), of a lanthanide oxohydroxide, of a mixed lanthanide oxohydroxide (i.e. of at least two lanthanides) or one of their mixtures.

The coating, when it is present, consists of at least one inorganic or mixed inorganic/organic constituent.

As this will become apparent from the description and the examples which follow, this coating, when it is present, advantageously consists of a polysiloxane (which is an inorganic constituent) but may also comprise organic molecules either directly attached to the surface of the core, or grafted on an inorganic coating, or even comprised within this inorganic coating.

The particles used according to the invention as exposed earlier, may only consist of oxides and/or oxohydroxides of lanthanides.

They may also be in a purely inorganic form, and comprise in this case a core consisting of oxides and/or oxohydroxides of lanthanides and coated with an inorganic coating.

They may also be in the form of inorganic and organic hybrid nanoparticles and consist of a core in oxides and/or oxohydroxides of at least one lanthanide and of a mixed organic/inorganic coating.

The hydrodynamic size of the particle is measured in laser granulometry by photon correlation spectroscopy. In the case when the particle is composite, the size of each of the successive layers is measured by this technique after each elaboration step. The real size of the particle and of its different constituents is measured by transmission electron microscopy. The sizes given in the present document unless indicated otherwise are the sizes measured in transmission electron microscopy.

The particles are preferentially in the core-shell form and preferably substantially spherical.

More generally, the lanthanide oxide or oxohydroxide core may be spherical, faceted (the dense planes then forming the interfaces) or of an elongated shape. Then also, the final particle is spherical, faceted or of an elongated shape.

The core or the actual nanoparticle when there is no coating advantageously, is between 1 and 30 nm.

According to the invention, particles of small dimensions will advantageously be used.

Indeed, particles of small dimensions generally have better colloidal stability and are more adapted to injections.

Their small size and their great stability allows better diffusion:
from the vascular compartment towards the target cells, by passing through the non-limited capillary barrier (due to their small size) and with high diffusion through the interstitial tissues right up to the cells, after intra-vascular injection.
tissue diffusion of small particles is high, responsible for large tissue retention after passing the capillary barrier.

As tumors are most often hypervascularied (as compared with healthy tissues), even in the absence of specific functionalization of the particles, a higher tissue capture is obtained at tumors as confirmed by the conducted MRI studies (see examples).

Comparatively with molecules for which elimination may also be accomplished through the kidneys, small size particles have a longer dwelling time inside the body and allow preferential accumulation in zones of interest (cf. Example 8 showing that by mass, 100 times more lanthanides as complexes than as nanoparticles are needed for having the same effect).

The small size of the particles allows urinary elimination of the particles (unlike larger particles), allowing rapid elimination of the fractions of particles not captured at the tumoral tissues. It limits non-specific capture by cells of the reticulo-endothelial system. This very favorable biodistribution was confirmed by MRI studies.

After absorption of an X or gamma ray, the lanthanides may emit secondary electrons (with an energy of a few keV), Auger or Compton electrons (with an energy of a few keV for X-rays having an energy below 100 keV). The small size of the particle allows escape of the electrons even if the excited lanthanide is found at the centre of the particle [Auger electrons crossing with negligible absorption a thickness of a few nanometers and secondary and Compton electrons a thickness of about 10 nm].

The small size nanoparticle thus allows:
a long dwelling time and increased permeation in the lesioned zones (unlike molecules)
facilitated kidney elimination (relatively to large size particles)
increased ionizing treatment efficiency (relatively to large size particles)
less sensitivity to standard elimination systems for example the liver by opsonizations (relatively to large size particles).

For all the reasons stated above and, as shown by the results presented in the examples, according to the present invention, nanoparticles are preferably used which have dimensions less than 5 nm and preferably less than 2 nm, when these are particles exclusively consisting of at least one oxide and/or one oxohydroxide of at least one lanthanide or having a core of dimensions less than 5 nm, and preferably less than 2 nm, when this is a particle in the form of a core and coating.

According to a particularly advantageous alternative of the invention, the portion of the nanoparticle containing the oxides and/or the oxohydroxides will comprise at least two different lanthanides, each accounting for more than 10% by mass of the totality of the lanthanides and preferably more than 20%.

This alternative has a most particular advantage when it allows adaptation of the particle to the X or gamma ray source.

Indeed, many irradiation sources are not monochromatic but polychromatic (compact source, poly- or mono-chromatic synchrotron radiation). It may therefore be interesting to adapt the lanthanide composition to the energy dispersion of the radiation, so as to absorb the whole of the spectrum and not only a particular energy.

The selection of the nature and of the proportion of the lanthanides (the threshold energy of which varies from 39 keV for lanthanum to 63 keV for lutetium) will be made in order to absorb the intensity maxima of the radiation.

In such a case when several different lanthanides will be used, the oxides and/or oxohydroxides of these different lanthanides may either be in successive layers, or as a solid solution.

The solid solution is easier to make from the point of view of synthesis. Moreover a multilayer structure may be interesting if the intention is to increase the contrast in magnetic resonance imaging (by putting the high contrast lanthanides at the surface) or if for better efficiency of the treatment, the lanthanides emitting lower energy electrons are arranged at the surface.

By selecting lanthanides which all have very close chemistries it is possible to make adaptable solid solutions easily.

One of the advantages of the use of nanoparticles of the invention is that, as this is well known, a certain number of lanthanides give signals in magnetic resonance imaging (MRI), which, provided proper selection of the proportions and nature of the lanthanides, allows an MRI signal to be produced during the therapy, allowing in vivo detection of the presence of particles and/or monitoring of the therapy.

Thus, the lanthanides will advantageously contain at least 50% by mass of gadolinium (Gd), of dysprosium (Dy), of holmium (Ho) or mixtures of these lanthanides.

The combination of a therapeutic effect and of in vivo imaging detectability properties is a major asset of the therapeutic agents.

Indeed, in vivo imaging will allow quantification of the tumoral tissue capture of the nanoparticles, establishment of the capture kinetics for being informed on the delay after injection when the latter are maximum in the tumor. With this information, it is possible to optimally determine the delay within which external irradiation should be performed, in order to obtain a maximum effect (maximum radiosensitizing effect). External detection by imaging of these nanoparticles therefore allows the therapy to be monitored.

These nanoparticles are responsible for a positive modification of the MRI contrast which may be quantified. Magnetic resonance imaging allows high resolution images to be obtained without any iatrogenic effect.

According to a particularly interesting alternative of the invention when the radiotherapy method is coupled with tracking by MRI, nanoparticles are used in which the portion containing oxides and/or oxohydroxides of lanthanides contains at its periphery lanthanides causing an MRI signal, preferably gadolinium, and at least one other lanthanide in its central portion.

Indeed, as MRI contrast is accomplished by action of the paramagnetic ion in the perimeter of its first coordination layers, it is preferable to put the lanthanides which will act as contrast agents at the surface of the core.

Lanthanides with a large atomic number which absorb radiations will then preferentially be located at the centre of this core. In order that Auger electrons generated by the absorption of the radiation may be extracted from the particle (escape distance of the order of one nanometer), it is therefore desirable that the latter be of small size.

Finally, according to another advantageous alternative of the invention, related to the fact that it is known that a certain number of lanthanides have capture cross-sections allowing their use in treatments by neutron therapy, a lanthanide may, if need be, in the contemplated therapy, be selected having a sufficient capture cross-section for this purpose, in order to also allow treatment by neutron therapy.

For this purpose, selection of gadolinium proves to be particularly interesting and lanthanides consisting of at least 50% by mass of gadolinium will advantageously be used, especially if the intention is to couple radiotherapy treatment with neutron therapy treatment.

Finally, oxides and/or hydroxides of lanthanides comprising at least 30% by mass of lutetium or ytterbium oxides or one of their mixtures will advantageously be used.

The interaction probability will thus be increased, which is directly linked to the density and the high atomic number of the interacting elements. The use of lutetium, the lanthanide with the highest atomic number, and of the oxide of which is the most dense, is thus particularly advantageous.

As stated earlier, the nanoparticles used according to the invention, are advantageously in the form of a core and of a coating.

This coating may either be inorganic or organic and inorganic.

According to an alternative of the invention, a coating consisting of polysiloxane will be used, on which molecules, in a particularly advantageous way hydrophilic organic molecules, are attached.

As discussed earlier, the coating may be an inorganic or mixed organic coating. Generally in the case when the nanoparticles have a coating, it is advantageous that the oxides and/or oxohydroxides of lanthanides account for at least 30% by mass relatively to the whole of the inorganic constituents of said nanoparticle, the organic constituents being covalently bound to the hybrid nanoparticle and representing less than 30% by mass of the final particle.

It will be noted that when reference is made to <<the whole of the inorganic constituents>> the oxides and oxohydroxides of lanthanides are included in this whole.

Generally, the function of the coating is multiple.

The question is of protecting the core from untimely dissolution on the one hand and of easily adapting the stability of the nanoparticles to the injection system (avoid uncontrolled agglomerations) because of the numerous surfactants which it is possible to attach on its surface, on the other hand.

Finally, another function of the coating is to adapt the charges and the surface chemistry in order to improve biodistribution and to promote local overconcentrations in the tumoral zones.

For this purpose, the polysiloxane, a coating selected within the scope of the invention, is particularly advantageous. The coating may exclusively consist of polysiloxane or exclusively of polysiloxane and organic molecules.

According to a particularly advantageous alternative, the nanoparticles used according to the present invention are in coated form and the coating consists of polysiloxane which accounts for 1 to 70% by mass of the inorganic constituents of the nanoparticle.

This coating in the form of polysiloxane is advantageously such that the number of silicon atoms relatively to the number of atoms of lanthanides is comprised between 0.1 and 8.

By selecting this ratio, it is possible to efficiently protect the core of the nanoparticle and/or adapt its colloidal stabilization and/or adapt its magnetic interaction with water and consequently its characteristics of contrast agents.

As explained earlier, the coating advantageously is a coating in polysiloxane and further comprises hydrophilic organic molecules with molar masses of less than 5,000 g/mol, preferably less than 800 g/mol, preferably still less than 450 g/mol.

These organic molecules advantageously comprise the following units given below with their preferred molar mass (Mw):
Polyethylene glycol, bis(carboxymethyl) ether (PEG), 250<Mw<5,000 g·mol⁻¹.
Polyoxyethylene bis(amine), 250<Mw<5,000 g·mol⁻¹.
O-methyl-O′-succinylpolyethylene glycol, Mw of the order of 2,000 g·mol⁻¹.
Methoxypolyethylene glycol amine (Mw of the order of 750 g·mol⁻¹)
Diethylenetriamine-pentaacetic acid (DTPA) and its derivatives, in particular dithiol DTPA (DTDTPA)
1,4,7,10-tetraazacyclodecane-1,4,7,10-tetraacetic acid (DOTA) and derivatives
Succinic and mercaptosuccinic acid
Glucose and derivatives, for example dextran

Hydrophilic amino acids or peptides (aspartic acid, glutamic acid, lysine, cysteine, serine, threonine, glycine . . . )

More generally, the organic molecules will advantageously comprise alcohol or carboxylic acid or amine or amide or ester or ether-oxide or sulfonate or phosphonate or phosphinate functions and will be preferably bound covalently to at least 10% of the silicon atoms of said polysiloxane.

Organic molecules comprising a polyethylene glycol, DTPA, DTDTPA (dithiol DTPA) unit or succinic acid will preferably be selected.

From the organic molecules above, those having complexing properties of lanthanides, in particular those for which the complexation constant is greater than 10¹⁵, will be advantageously selected.

For this purpose, DTPA or DOTA will be preferably selected.

It will be easily understood that depending on the targeted application, in particular depending on the nature of the tumor to be treated, on the injection technique and on the radiation dose, the proportion of oxides and/or oxohydroxides of lanthanides may vary in wide proportions. However, generally, the compositions used according to the invention will advantageously contain between 0.5 and 200 g/L of oxide and/or oxohydroxides of lanthanides.

The composition will be injected into the body either directly into the tumor to be treated or via a parenteral route, in particular an intravenous route.

Irradiation by standard techniques of radiotherapies or Curie-therapy will then be achieved either directly after injection or after a determined time in order to have maximum efficiency and selectivity between healthy tissue and marked tissue. This waiting time and the irradiation dose may advantageously be determined by observing the positioning and fate of the nanoparticles after injection, for example by MRI.

Among the nanoparticles described above and which may be advantageously used within the scope of the invention, as discussed earlier, a certain number of them are novel and are novel products per se.

These are nanoparticles in the coated form and comprising a core for which the size is less than 2 nm and, preferably, comprised between 1 and 2 nm.

These nanoparticles comprise a coating consisting of polysiloxane, the nanoparticle comprising 1 to 5 silicon atoms per lanthanide and, at least 10% of the silicon atoms are bound to hydrophilic organic molecules with molar masses of less than 450 g/mol, preferably selected from organic molecules including alcohol or carboxylic acid or amine or amide or ester or ether-oxide or sulfonate or phosphonate or phosphinate functions, covalently bound to at least 10% of silicon atoms of said polysiloxane.

According to an advantageous alternative, said hydrophilic organic molecules contain a polyethylene glycol, DTPA, DTDTPA (dithio DTPA) unit or succinic acid.

According to another advantageous alternative, said hydrophilic organic molecules are complexing agents of lanthanides, the complexation constant is greater than 10¹⁵, preferably DTPA or DOTA.

These organic molecules will advantageously be selected from those having one or more carboxylic acids, for example DTPA and its derivatives.

It will be noted that all these novel products are part of those for which the use is preferred within the scope of the invention.

Indeed, even if all the nanoparticles which may be used according to the invention prove to be particularly useful as radiosensitizing agents, notably because of the presence of oxides and/or oxohydroxides of lanthanides which efficiently interact with X or gamma energetic radiations, the coated particles having a core of very reduced dimensions and comprising a polysiloxane coating, and preferably a polysiloxane coating, on which are grafted organic molecules such as those defined earlier, prove to be particularly efficient for increasing the therapeutic efficiency of a treatment with X or gamma rays, as this is apparent from the examples hereafter.

Hybrid nanoparticles and most particularly those for which the core is particularly fine, preferably with dimensions of less than 5 nm, and still preferably comprised between 1 and 2 nm, make it possible to obtain very large amplification of the dose effect, even larger than what is observed with gold nanoparticles, as this is apparent from the examples hereafter.

The presence of a polysiloxane layer after injection in vivo, imparts to the nanoparticle a suitable behavior for the sought irradiation goal.

Moreover, the very small size of the core and of the coating, depending on the functionalization, makes it possible to obtain sufficiently stable and controllable particles for intravenous or directly intratumoral injections in vivo.

Generally, the nanoparticles used according to the invention, and more particularly the novel nanoparticles, make it possible to locally obtain an overconcentration at the tumor. These particles may be designed so as to locally have an overconcentration at the tumor (either directly linked to increase of the blood irradiation zone, or linked to passive diffusion within the tumor, or by functionalization with active biomolecules).

A great benefit of the method comes from the preferential use of nanoparticles of oxidized lanthanides having interesting magnetic properties and which may be directly tracked by MRI imaging.

With this technique it is then possible to optimize the moment between the injections and the therapies. The zones to be irradiated may thereby also be localized at best.

Another great benefit of the method consists of using cores based on mixed oxide lanthanides, i.e. containing several different lanthanides or using a mixture of hybrid nanoparticles with different cores.

With this approach, it is thereby possible to adapt at best the radiosensitizer to the energy spectrum of the irradiation source, to the therapy and to the patient.

Generally, one skilled in the art may easily make particles as used according to the invention, and in particular novel particles, by relying on different literature documents.

More specifically, as regards the synthesis of the different nanoparticles used according to the invention, the following elements will be noted:

the oxides and oxohydroxides of lanthanides are preferentially made according to methods using a polyol as a solvent, for example according to the approach described in the publication of R. Bazzi et al. (Bazzi, R.; Flores, M. A.; Louis, C.; Lebbou, K.; Zhang, W.; Dujardin, C.; Roux, S.; Mercier, B.; Ledoux, G.; Bernstein, E.; Perriat, P.; Tillement, 0. Synthesis and properties of europium-based phosphors on the nanometer scale: Eu203, Gd203:Eu, and Y203:Eu. Journal of Colloid and Interface Science (2004), 273(1), 191-197.) or in the publication of M. Flores et al. (Flores-Gonzalez, M.; Ledoux, G.; Roux, S.; Lebbou, K.; Perriat, P.; Tillement, 0.; Preparing nanometer scaled Tb-doped Y203 luminescent powders by the polyol method. Journal of Solid State Chemistry (2005),178, 989-997.). They may also be made by synthesis, freeze-drying and partial decomposition heat treatment, as described in the publication of C. Louis et al. (Louis, C.; Bazzi, R.; Flores, M. A.; Zheng, W.; Lebbou, K.; Tillement, O.; Mercier, B.; Dujardin, C.; Perriat, P. Synthesis and characterization of Gd2O3:Eu3+ phosphor nanoparticles by a sol-lyophilization technique. Journal of Solid State Chemistry (2003), 173(2), 335-341.).
For the coating with layers of polysiloxanes, several techniques may be used, derived from those initiated by Stoeber (Stoeber, W; J. Colloid. Interf. Sci. 1968, 26, 62). The method used for coating particles may also be used, as shown in the publication of C. Louis et al. (Louis, C.; Bazzi, R.; Marquette, C.; Bridot, J. L.; Roux, S.; Ledoux, G.; Mercier, B.; Blum, L.; Perriat, P.; Tillement, O.; Nanosized hybrid particles with double luminescence for biological labeling, Chemistry of Materials (2005), 17, 1673-1682.) and International Application WO 2005/088314.
For the organic grafting at the surface, it is recommended to directly use silanes containing hydrophilic organic functions or to proceed in two steps by grafting compounds on the aminated surface for example in the case of the use of APTES.
Functionalization at the surface of the coated nanoparticles of lanthanide oxide aims i.a. at introducing hydrophilic molecules in order to promote free circulation of nanoparticles in the vascular system and to thereby avoid an undesirable non-specific accumulation. One of the possible strategies consists of inserting into the polysiloxane layer (during its formation) organoalkoxy silanes, the organic portion of which is hydrophilic, for example PEG chains terminated by an alkoxysilyl group, a glucose derivative or a phosphonate group bound to an alkoxysilyl group. Another way of making the particles perfectly circulating consists of post-functionalizing the polysiloxane layer with hydrophilic molecules having a reactive group capable of ensuring the attachment of the molecule on the polysiloxane layer which should have grafting sites. Therefore it is necessary in order to make this polysiloxane layer, to use organoalkoxy silanes having reactive groups such as derivatives of succinic anhydride, amine, thiol, maleimide, carboxylic acid, isocyanate, isothiocyanate, epoxide groups, to which the hydrophilic molecules will be attached by a covalent bond (following a condensation reaction). For the most part, hydrophilic entities include a hydrocarbon chain with at least one ionizable (carboxylic acid, amine, phosphate, phosphinate, phosphonate, sulfonate) function and/or several alcohol and/or ether-oxide and/or thiol functions.

As regards treatment by radiotherapy, one skilled in the art will easily understand that the source will be adapted to the nature of the nanoparticle as well as to the type of tumor to be treated.

Thus, the use of gadolinium within a therapeutic context requires the use of a source of X-rays having a critical energy located above the absorption threshold K of gadolinium (53.4 keV). This source may be monochromatic in order to be selected for the relevant element, here gadolinium (SSRT of ESRF technique) and this seems to be the best configuration. It may also be polychromatic (white beam of ESRF) and be used in the MRT mode (splitting of the beam into microbeams and depositing a dose of the order of 600 Gy). Further, more conventionally, gadolinium should also be interesting if it is used with a conventional adapted spectrum irradiator.

### EXAMPLES

### Example 1

### Synthesis of the Gadolinium Oxide Core

A colloid is prepared by dissolving an amount of 56 g·L⁻¹ of gadolinium chloride salts in a volume of 1 L of diethylene glycol. To the obtained solution, addition of 45 mL of soda at a concentration of 3M is performed at room temperature within 1 h 30 min. The mixture is then heated to 180° C. for 4 hrs. The final size of the particles, as measured by granulometry, is about 1.5 nm.

Around these particles, a layer of functionalized polysiloxane with a thickness of 0.5 nm is synthesized via a sol-gel route.

In a solution containing 200 mL of colloid and 800 mL of diethylene glycol, 3.153 mL of amino-propyltriethoxysilane (APTES), 2.008 mL of tetra-ethylorthosilicate (TEOS) and 7.650 mL of a triethylamine 0.1M aqueous solution are added.

The reaction is conducted at 40° C. in an oil bath and under stirring in several steps.
at t=0 h addition of 3% of the APTES and TEOS volume
at t=1 h addition of 3% of the total water volume
at t=2 h addition of 7% of the APTES and TEOS volume
at t=3 h addition of 7% of the total water volume
at t=4 h addition of 10% of the APTES and TEOS volume
at t=5 h addition of 10% of the total water volume
at t=6 h addition of 15% of the APTES and TEOS volume
at t=7 h addition of 15% of the total water volume
at t=23 h addition of 15% of the APTES and TEOS volume
at t=24 h addition of 15% of the total water volume
at t=25 h addition of 25% of the APTES and TEOS volume
at t=26 h addition of 25% of the total water volume
at t=27 h addition of the remaining 25% of the APTES and TEOS volume
at t=28 h addition of the remaining 25% of the total water volume
at t=76 h end of the synthesis

The thereby coated particles are then functionalized:
either with poly(ethylene glycol) bis(carboxymethyl) ether (Mₙ=250) (PEG250):

0.3612 g of N-(3-dimethylaminopropyl)-N′-ethyl-carbodiimide, 0.3489 g of pentafluorophenol and 181.5 μL of PEG250 are dissolved in 5 mL of isopropanol under stirring for 1 h 30 min. The obtained solution is then mixed with 100 mL of a solution of coated nanoparticles. The mixture is then stirred for 15 hrs.

FIG. 1 gives the measurement by photon correlation spectroscopy (PCS, with a size analyzer Zetasizer NonoS Malvern Instrument) of the size and size distribution of the nanoparticles prepared according to this example, and more specifically:
Curve I: gadolinium oxide nanoparticles before coating:
Curve II: gadolinium oxide nanoparticles after coating with a polysiloxane layer;
Curve III: after grafting PEG250 on the grafting sites (amine of APTES) of the polysiloxane layer.
Or with diethylenetriamine-pentaacetic acid (DTPA):

0.616 g of DTPA are dissolved in 17.3 mL of anhydrous dimethylsulfoxide (DMSO) under stirring for 1 h 30 min. The obtained solution is then mixed with 100 mL of solution of coated nanoparticles. The mixture is then stirred for 15 hrs.

### Example 2

### Synthesis of the Holmium Oxide Core

A colloid is prepared by dissolving an amount of 56 g·L⁻¹ of holmium chloride salts in a volume of 200 mL of diethylene glycol. To the obtained solution, addition of 7.5 mL of soda at a concentration of 3M is performed at room temperature within 1 h 30 min. The mixture is then heated to 180° C. for 4 hrs. The final size of the particles, as measured by granulometry, is about 1.5 nm.

Around these particles, a functionalized polysiloxane layer with a thickness of 0.5 nm is synthesized via a sol-gel route.

In a solution containing 200 mL of colloid and 800 mL of diethylene glycol, 3.153 mL of aminopropyl-triethoxysilane (APTES), 2.008 mL of tetraethyl-orthosilicate (TEOS) and 7.650 mL of a triethylamine 0.1M aqueous solution are added.

The reaction is conducted at 40° C. in an oil bath and under stirring in several steps.
at t=0 h addition of 3% of the APTES and TEOS volume
at t=1 h addition of 3% of the total water volume
at t=2 h addition of 7% of the APTES and TEOS volume
at t=3 h addition of 7% of the total water volume
at t=4 h addition of 10% of the APTES and TEOS volume
at t=5 h addition of 10% of the total water volume
at t=6 h addition of 15% of the APTES and TEOS volume
at t=7 h addition of 15% of the total water volume
at t=23 h addition of 15% of the APTES and TEOS volume
at t=24 h addition of 15% of the total water volume
at t=25 h addition of 25% of the APTES and TEOS volume
at t=26 h addition of 25% of the total water volume
at t=27 h addition of the remaining 25% of the APTES and TEOS volume
at t=28 h addition of the remaining 25% of the total water volume
at t=76 h end of the synthesis

The thereby coated particles are then functionalized:
either with poly(ethylene glycol) bis(carboxymethyl) ether (Mₙ=250) (PEG250):

0.3612 g of N-(3-dimethylaminopropyl)-N′-ethyl-carbodiimide, 0.3489 g of pentafluorophenol and 181.5 μL of PEG250 are dissolved in 5 mL of isopropanol under stirring for 1 h 30 min The obtained solution is then mixed with 100 mL of a solution of coated nanoparticles. The mixture is then stirred for 15 hrs.
Or with diethylenetriamine-pentaacetic acid (DTPA):

0.616 g of DTPA are dissolved in 17.3 mL of anhydrous dimethylsulfoxide (DMSO) under stirring for 1 h 30 min. The obtained solution is then mixed with 100 mL of solution of coated nanoparticles. The mixture is then stirred for 15 hrs.

### Example 3

### Synthesis of Gd/Ho (50/50) Mixed Oxide

A colloid is prepared by dissolving an amount of 56 g·L⁻¹ of holmium and gadolinium chloride salts in a volume of 200 mL of diethylene glycol. To the obtained solution, addition of 7.5 mL of soda at a concentration of 3M is performed at room temperature within 1 h 30 min. The mixture is then heated to 180° C. for 4 hrs. The final size of the particles, as measured by granulometry, is about 1.3 nm

In a solution containing 40 mL of colloid and 160 mL of diethylene glycol, 0.421 mL of aminopropyltriethoxy silane (APTES), 0.267 mL of tetraethylorthosilicate (TEOS) and 1.020 mL of a triethylamine 0.1M aqueous solution are added.

The reaction is conducted at 40° C. in an oil bath and under stirring in several steps.
at t=0 h addition of 10% of the APTES and TEOS volume
at t=1 h addition of 10% of the total water volume
at t=2 h addition of 25% of the APTES and TEOS volume
at t=3 h addition of 25% of the total water volume
at t=4 h addition of the remaining 65% of the APTES and TEOS volume
at t=5 h addition of the remaining 65% of the total water volume
at t=53 h end of the synthesis

### Example 4

### Synthesis of Oxide Nanoparticles of about 50 nm

A colloid is prepared by dissolving an amount of 56 g·L⁻¹ of gadolinium chloride salts in a volume of 200 mL of diethylene glycol. To the obtained solution, addition of 30 mL of soda at a concentration of 3M is performed at room temperature within 2 hrs.

The size of the obtained particles, as measured by granulometry, is about 50 nm.

### Example 5

### Formulation of Injectable Solutions

The lanthanide oxide particles of Examples 1-3 are purified by a succession of dialyses, against a mixture of diethylene glycol and ethanol with an increasing proportion of ethanol (up to 100%) and the volume of which is about twenty times greater than that of the solution to be purified. Purification is validated by elementary analysis by ICP-MS.

After purification, the colloid solution is re-concentrated in PEG400A (poly(ethylene glycol) of molecular mass 400 g·mol⁻¹) by adding a defined volume of PEG400 and removing the ethanol under reduced pressure. The thereby preserved particles may be stored for several months at a high concentration (up to 200 mM).

Injectable solutions are prepared by diluting the PEG400 solutions with a high concentration of nanoparticles, with a solution of HEPES and NaCl in order to finally obtain a HEPES and NaCl concentration of 10 and 145 mM, respectively. The nanoparticle concentration is such that [Gd] is comprised between 0.1 and 25 mM.

For example, to 0.2 ml of a solution of gadolinium oxide nanoparticles prepared according to Example 1, and concentrated in PEG400 ([Gd]=85 mM), are added portionwise 3.2 mL of an aqueous solution containing 10.6 mM of HEPES and 154 mM of NaCl. After each addition, the solution is vigorously stirred. After injection, the solution is filtered through membranes, the pore size of which is 220 nm.

### Example 6

### Implantation of Brain Tumors to Rats, Imaging and Survival

Implantation of brain tumors (gliosarcomas) is carried out fifteen days before the treatment by stereotaxic injection of 9 L cells (10⁴ cells in 1 μL ) at the right caudate nucleus of 300 g Fisher rats (coordinates: 3.5-5.5). An imaging examination by magnetic resonance (MRI) conducted fifteen days before implantation with an imager 7T (Biospec System 70/20, Brucker, Ettlingen, Germany) reveals, on the weighted images T₂ (TE=76.3 ms, TR=4,488 ms), cut thickness=0.6 mm, spatial resolution=0.0117*0.0156 cm/pixel), the presence of the tumor which is however not visible on the weighted images T₁ (TE=5.6 ms, TR=304.6 ms, cut thickness=0.6 mm, spatial resolution=0.0088×0.0130 cm/pixel) (cf. FIG. 2 which shows the weighted images T₂ of the brain of a rat bearing an tumor implanted according to this example). The pale grey area in the right portion of the brain delimits the location of the tumor.

Without any treatment, i.e. without any particle being administered and without any dose being delivered, the rats bearing this kind of tumors on average die after 19 days.

### Example 7

### Rat Brain MRI after Injection via an Intratumoral Route of Gadolinium Oxide Nanoparticles

15 μL of an injectable solution prepared according to Example 5 ([Gd]=5 mM) were directly injected at the centre of the tumor (i.e. reusing the trepanning site used upon injecting the cells) at a rate of 30 μL/h. While it was demonstrated that gadolinium oxide nanoparticles generate positive contrast which is expressed by a lightening of the area where the nanoparticles have accumulated, the examination of weighted images T₁ (TE=5.6 ms, TR=304.6 ms), cut thickness=0.6 mm, spatial resolution=0.0088*0.0130 cm/pixel), obtained after the injection (between 2 and 40 minutes after injection) reveals darkening of the image in the tumoral area with whitening at the periphery. This darkening results from high concentration of paramagnetic element: the particles have become strongly concentrated in the tumoral area with a decrease of the concentration from the centre towards the periphery. The intratumoral injection of these particles therefore facilitates the viewing of the tumor on T₁ images while it is invisible in the absence of nanoparticles (cf. FIG. 4 which shows the weighted images T₁ of the brain of a rat after intratumoral injection of an injectable solution of gadolinium oxide nanoparticles (according to this example).

### Example 8 (Comparative)

### MRI of Rat Brain after Injection of Gadolinium Complexes via an Intratumoral Route

15 μL of a commercial solution of gadolinium complexes ([Gd]=500 mM) were directly injected at the centre of the tumor (i.e. reusing the trepanning site used upon injecting the cells) at a rate of 30 μL/h. This solution is very widely used for clinical examinations. Examining the weighted images T₁ (TE=5.6 ms, TR=304.6 ms, cut thickness=0.6 mm, spatial resolution=0.0088*0.0130 cm/pixel), obtained after the injection (between 2 and 40 minutes after injection) reveals darkening of the image in the tumoral area with whitening at the periphery. This darkening results from high concentration of paramagnetic element: the molecules have become strongly concentrated in the tumoral area with a decrease of the concentration from the centre towards the periphery. The intratumoral injection of these molecules therefore facilitates the viewing of the tumor on T₁ images while it is invisible in the absence of nanoparticles. It should however be noted that the gadolinium concentration is 100 times greater than that of the colloidal solutions of gadolinium oxide hybrid nanoparticles which have been injected (Example 7).

### Example 9

### MRI of Brain Rat after Injection of Gadolinium Oxide Nanoparticles via an Intravenous Route

1.4 μL of an injectable solution prepared according to Example 5 ([Gd]=5 mM) were injected via an intravenous route in a caudal vein of a rat bearing an implanted tumor according to Example 6. The weighted images T₁ obtained 20 minutes after intravenous injection of the nanoparticles reveal a slight enhancement (whitening) in the area of the tumor which is however sufficiently significant so that it is not ambiguous (contrast enhancement: 15%). The whitening results from a stronger concentration of nanoparticles in the tumoral area than in the healthy tissue. Such a difference may be explained by greater vascularization of the tumor than that of the healthy tissue and/or by passive accumulation of particles in the tumor because of increased porosity of the blood vessels irrigating the tumors.

FIG. 5 shows the weighted images T₁ of the brain of a rat before (FIG. 5a) and after (FIG. 5b) intravenous injection of an injectable solution of gadolinium oxide nanoparticles according to this example. The whitening is marked by an arrow.

### Example 10

### Treatment of Rats Bearing a Tumor by X Microbeams

The rats bearing a tumor implanted according to Example 6 were irradiated in the MRT mode (microbeam radiation therapy) with a dose at the skin of 625 Gy in a unidirectional shoot of 51 microbeams, the width of which was 25 microns and the spacing 200 microns.

FIG. 3 shows the survival curve of rates bearing a gliosarcoma (implanted according to Example 6); curve I is obtained for the rats of Example 6 (without irradiation). Curve II corresponds to rats treated according to the present example (with irradiation).

The sick rats which were not treated all died 19 days after implantation, while the rats having been irradiated survive for a longer time.

The thereby treated rats survive longer than non-irradiated rats as shown by the displacement of the survival curve of FIG. 3 towards the right. However, all the rats die less than thirty days after implantation.

### Example 11

### Treatment of Rats Bearing a Tumor by X Microbeams after Intratumoral Injection of a Solution of Gadolinium Chelates

Twenty minutes after intratumoral injection in the centre of the tumor (i.e. by reusing the trepanning site used upon injecting the cells) of 15 μL of a commercial solution of gadolinium chelates ([Gd]=500 mM), the rats bearing a tumor (implanted according to Example 6) were irradiated in a crossed beam mode with a dose at the skin of 460 Gy for each beam of the 51 microbeams, the width of which was 25 μm and the spacing 200 μm. The irradiation field used was 13×10. Significant improvement in survival is noticed since about 33% of the rats survive 45 days after implantation.

### Example 12 (Comparative)

### Treatment of Rats Bearing a Tumor by X Microbeams after Intratumoral Injection of a Solution of Gold Nanoparticles Covered with Gadolinium Chelates (Au@TDDTPA-Gd)

Twenty minutes after intratumoral injection at the centre of the tumor (i.e. by reusing the trepanning site used upon injecting the cells) of 15 μL of an injectable solution of gold nanoparticles (Au@DTDTPA-Gd) prepared according to the procedure described by Debouttière et al. (Adv. Funct. Mater. 2006, 16, 2330-2339 ([Au]=45 mM and [Gd]=5 mM), the rats bearing a tumor (implanted according to Example 6) were irradiated in a crossed beam mode with a dose at the skin of 460 Gy for each beam of the 51 microbeams, the width of which was 25 μm and the spacing 200 μm. The irradiation field used was 13×10. Improvement in survival is noticed since about 25% of the rats survive 40 days after implantation. However, all the rats died beyond 45 days after implantation.

FIG. 6 shows the survival curve of rats bearing a gliosarcoma (implanted according to Example 6) after intratumoral injection of Au@DTDTPA-Gd nanoparticles (curve II) and irradiation by X microbeams (according to the present example) as compared with non-treated controls (curve I).

### Example 13 (Comparative)

### Treatment of Rats Bearing a Tumor by X Microbeams after Intravenous Injection of a Solution of Gold Nanoparticles Covered with Gadolinium Chelates (Au@TDDTPA-Gd)

Twenty minutes after intravenous injection of 1.4 mL of an injectable solution of gold nanoparticles Au@DTDTPA-Gd prepared according to the procedure described by Debouttière et al. (Adv. Funct. Mater. 2006, 16, 2330-2339 ([Au]=45 mM and [Gd]=5 mM) in one of their caudal veins, the rats bearing a tumor (implanted according to Example 6) were irradiated in a crossed beam mode with a dose of the skin of 460 Gy for each beam of the 51 microbeams, the width of which was 25 μm and the spacing 200 μm. The irradiation field used was 13×10. Improvement in survival is noticed since about 33% of the rats survive 45 days after implantation. However, all the rats died beyond 50 days after implantation.

### Example 14

### Treatment of Rats Bearing a Tumor by X Microbeams after Intratumoral Injection of a Solution of Gadolinium Oxide Nanoparticles

Twenty minutes after intratumoral injection at the centre of the tumor (i.e. by reusing the trepanning site used upon injecting the cells) of 15 μL of an injectable solution of gadolinium oxide nanoparticles prepared according to Example 5 ([Gd]=5 mM), the rats bearing a tumor (implanted according to Example 6) were irradiated in a crossed beam mode with a dose at the skin of 460 Gy for each beam of the 51 microbeams, the width of which was 25 μm and the spacing 200 μm. The irradiation field used was 13×10.

A significant improvement in survival is noticed since about 25% of the rats survive 45 days after implantation (FIG. 7).

### Example 15

### Treatment of Rats Bearing a Tumor by X Microbeams after Intravenous Injection of an Intravenous Solution of Gadolinium Oxide Nanoparticles

Twenty minutes after intratumoral injection of 1.4 mL of an injectable solution of gadolinium oxide nanoparticles prepared according to Example 5 ([Gd]=5 mM), the rats bearing a tumor (implanted according to Example 6) were irradiated in a crossed beam mode with a dose at the skin of 460 Gy for each beam of the 51 microbeams, the width of which was 25 μm and the spacing 200 μm. The irradiation field used was 13×10. Significant improvement in survival is noticed since about 25% of the rats survive 45 days after implantation. However, all the rats died beyond 45 days after implantation.

FIG. 8 shows the survival curve of rats bearing a gliosarcoma (implanted according to Example 6) after intravenous injection of a solution of gadolinium oxide nanoparticles and irradiation by X microbeams according to this example (curve II, to be compared with the survival curve (I) without any treatment).

Note: Examples 10-15 show that the X-ray treatment improves the survival of animals as compared with non-treated animals (Example 6). This improvement is more significant when radiosensitizing agents are administered to the sick animals. It is important to notice that the use of gadolinium oxide nanoparticles coated with a layer of polysiloxane functionalized by a DTPA gives results similar to those of gadolinium complexes, probably due to a greater local concentration of the particles in the zone to be treated even if the solutions of complexes are 100 times more concentrated in gadolinium than the nanoparticle solutions. Even more surprisingly, the effect of the gadolinium oxide nanoparticles is greater than that of gold particles while the element gold (nine times more abundant in the injected solutions than gadolinium) is characterized by a higher atomic number Z. It will be noted that the effect is even greater, taking into account the fact that the rats which were treated with gold nanoparticles all died after 45-50 days whereas part of the rats treated with gadolinium oxide nanoparticles were still alive during the same period.

1. The use of nanoparticles with dimensions comprised between 1 and 50 nm, at least one portion of which consists of at least one oxide and/or one oxohydroxide of at least one lanthanide, said nanoparticles:
either consisting of a least one oxide and/or one oxohydroxide of at least one lanthanide,
or in the form of nanoparticles comprising a core consisting of at least one oxide and/or one oxohydroxide of at least one lanthanide, and a coating consisting of a polysiloxane, with possibly organic molecules grafted at the surface or comprised inside it,
as a radio-sensitizing agent in the making of an injectable composition intended to improve the efficiency of the treatment of a tumor by X or gamma irradiations.

2. The use according to claim 1, characterized in that said nanoparticle has dimensions of less than 5 nm, preferably less than 2 nm.

3. The use according to claim 1, characterized in that said nanoparticles contain oxides and/or oxohydroxides of at least two different lanthanides each accounting for more than 10% by mass of the totality of the lanthanides and preferably more than 20%.

4. The use according to claim 3, characterized in that the oxides and/or oxohydroxides of different lanthanides are in successive layers.

5. The use according to claim 3, characterized in that the oxides and/or oxohydroxides of different lanthanides are in the form of a solid solution.

6. The use according to claim 1, characterized in that said lanthanides comprise at lest 50% by mass of lanthanides producing a signal in MRI, allowing in vivo detection of the presence of said particles and/or monitoring of the therapy.

7. The use according to claim 5, characterized in that said lanthanides contain at least 50% by mass of gadolinium (Gd), of dysprosium (Dy), of holmium (Ho) or of mixtures of these lanthanides.

8. The use according to claim 1, characterized in that at least one portion of the lanthanides is selected in order to have a sufficient cross-section for neutron capture so as to also allow treatment by neutron therapy.

9. The use according to claim 1, characterized in that said oxides and/or hydroxides of lanthanides comprise at least 30% by mass of lutetium or ytterbium oxide, or of one of their mixtures.

10. The use according to claim 1, characterized in that said lanthanides comprise at least 50% gadolinium.

11. The use according to claim 1, characterized in that said portion containing at least one oxide and/or oxohydroxide of at least one lanthanide contains at its periphery lanthanides producing an MRI signal, preferably gadolinium and at least one other lanthanide in its central portion.

12. The use according to claim 1, characterized in that said nanoparticles have an inorganic or mixed inorganic/organic coating and in that said oxides and/or oxohydroxides of lanthanides account for at least 30% by mass relatively to the whole of the inorganic constituents of said nanoparticle, the organic constituents being covalently bound to the hybrid nanoparticle and accounting for less than 30% by mass of the final particle.

13. The use according to claim 1, characterized in that said nanoparticles comprise a core in the form of at least one oxide and/or oxohydroxide of at least one lanthanide and a polysiloxane coating accounting for 1 to 70% by mass of the inorganic constituents of said nanoparticle.

14. The use according to claim 13, characterized in that said coating is in the form of a polysiloxane and in that the silicon atom number/lanthanide atom number ratio is comprised between 0.1 and 8.

15. The use according to claim 13, characterized in that said coating is a polysiloxane coating and in that said nanoparticle further comprises hydrophilic organic molecules with molar masses of less than 5,000 g/mol and preferably less than 450 g/mol preferably selected from organic molecules including alcohol or carboxylic acid or amine or amide or ester or ether-oxide or sulfonate or phosphonate or phosphinate functions, covalently bound to at least 10% of the silicon atoms of said polysiloxane.

16. The use according to claim 15, characterized in that said hydrophilic organic molecules contain a polyethylene glycol, DTPA, DTDTPA (dithioly DTPA) unit or succinic acid.

17. The use according to claim 15, characterized in that said hydrophilic organic molecules are complexing agents of lanthanides, the complexation constant of which is greater then 10¹⁵, preferably DTPA or DOTA.

18. The use according to claim 1, characterized in that said composition contains between 0.5 and 200 g/L of oxide(s) and/or oxohydroxides of lanthanides.

19. The use according to claim 1, characterized in that said composition is in the form of an injectable colloidal dispersion.

20. Nanoparticles characterized in that they consist:
of a core consisting of at least one oxide and/or one oxohydroxide of at least one lanthanide as defined in any of the preceding claims, said core having a size comprised between 1 and 2 nm, and
of a coating of said core in polysiloxane comprising 1 to 5 silicon atoms per lanthanide and at least 10% of the silicon atoms of which are bound to hydrophilic organic molecules with molar masses of less than 450 g/mol, preferably selected from organic molecules including alcohol or carboxylic acid or amine or amide or ester or ether-oxide or sulfonate or phosphonate or phosphinate functions covalently bound to at least 10% of the silicon atoms of said polysiloxane.

21. The nanoparticles according to claim 20, characterized in that said hydrophilic organic molecules contain a polyethylene glycol, DTPA, DTDTPA (dithiol DTPA) unit or succinic acid.

22. The nanoparticles according to claim 20, characterized in that said hydrophilic organic molecules are complexing agents of lanthanides, the complexation constant of which is greater than 10¹⁵, preferably DTPA or DOTA.
